(19) United States
(12) Patent Application Publication
(10) Pub. No.: US 2022/0409075 A1
(43) Pub. Date: Dec. 29, 2022

(54) PHYSIOLOGICAL CONDITION MONITORING SYSTEM AND METHOD THEREOF

(71) Applicant: PANASONIC INTELLECTUAL PROPERTY MANAGEMENT CO., LTD., Osaka (JP)

(72) Inventors: Ramdas KRISHNAKUMAR, Singapore (SG); Muhammad USMAN, Singapore (SG); Pradeep RAJAGOPALAN, Singapore (SG); Ariel BECK, Singapore (SG); Khai Jun KEK, Singapore (SG); Yasufumi SHIRAKAWA, Osaka (JP)

(21) Appl. No.: 17/358,401

(22) Filed: Jun. 25, 2021

(51) Int. Cl. A61B 5/024; A61B 5/00; A61B 5/318

(52) U.S. Cl. CPC A61B 5/02405; A61B 5/7275; A61B 5/7225; A61B 5/318; A61B 5/7267

(57) ABSTRACT

A system (101) for monitoring a physiological condition of a user (104) is disclosed herein. The system (101) includes a receiving module (110) configured to receive a plurality of short-term segments of Heart Rate Variability (HMI) (302) or short-term electrocardiogram (ECG) segments (402) or short voice recordings (602) from the user (104) recorded at different time points. The system includes a stitching module (114) for stitching the plurality of short-term segments and creating a stitched segment. The system further includes an extracting module (116) extracting feature from the stitched segment and a predicting module (118) for predict the physiological condition, based on the feature.

### FIELD OF THE INVENTION

The present disclosure, in general, relates to a system and method for monitoring physiological condition, More particularly, the invention relates to a system and method for processing health indicator user signals for predicting physiological condition.

### BACKGROUND

With the analysis of electrocardiograms (ECGs), it has been possible to inform to a patient about an underlying cardiac disorder. The analysis may help in predicting probability of any abnormal heart condition, chronic diseases affecting heart condition such as hypertension. Skin surface ECG signal analysis is typically in accordance with waveform time domain parameters and may depend heavily on the quality of the signal received and analyzed. Thus, more reliable information can he extracted from the recorded waveform if higher quality ECG is recorded. The waveforms may be analyzed post filtering and cleaning of the signal for various indicators useful to detect cardiac events or abnormal heart conditions, such as hypertension and its characterization. The analysis of indicators such as Heart Rate Variability (HRV), ECG signals, forms a vital indicator for predicting the underlying abnormal heart conditions. Other indicators to predict physiological condition of the patient experiencing stress, is through analyzing the mood of the patient. Moods are long lasting state that can be analysed over longer period of time via the voice of the patient.

Heart rate variability (HRV) signals are the variance in time between the beats of heart. These periods of time between successive heart beats are known as RR intervals based on the heartbeat's R-phase, spikes present in an electrocardiogram (ECG) and measured in milliseconds. A greater variability of HRV indicates that the body is more suited to execute at a high level.

However, there has always been a strict requirement for longer continuous HRV signal measurements. The duration of the RR interval series is not a matter of convenience but a fine balance between two important issues, i.e., an acceptable variance and stationarity of the time series on one aspect, and an acceptable resolution of the spectral estimate and reduced spectral leakage on the other aspect.

Furthermore, in a real-world for measurement of data, it is preferred to use smaller measurement intervals so that parameters governing measurements are better controlled. However, considering the patient with underlying conditions of hypertension over larger time intervals, smaller measurement intervals may cause under-sampling of data and thus results in poor performance of a system predicting the underlying condition of the patient. Additionally, the biological data such as ECG measurement often require the patient to sit still over long periods of time making it difficult for the medical practitioners to analyse the data for the patient and monitor and predict physiological conditions.

Thus, there is a need for a solution that overcomes the above limitation of state-of-the art solution and renders an effective and efficient system and method for monitoring physiological condition with non-ideal data span.

Thus, there is a need for a solution that determines physiological condition by processing HRV signals, ECG signals from the patient. Also, determine mood of the patient by analyzing physiologically relevant indicators such as voice.

### SUMMARY

This summary is provided to introduce a selection of concepts, in a simplified format, that are further described in the detailed description of the invention. This sum is neither intended to identify key or essential inventive concepts of the invention and nor is it intended for determining the scope of the invention.

A system for monitoring a physiological condition of a user is disclosed herein. The system includes a receiving module configured to receive plurality of short-term Heart Rate Variability (HRV) segments of the user. The plurality of short-term HRV segments is recorded at different time points. The system ncludes a stitching module in communication with the receivine module and configured to stitch the plurality of short-trm HRV segments is for creating a stitched HRV segment. The system also includes an extracting module in communication with the stitching module and configured to extract at least one feature from the stitched HRV segment, and a predicting module in communication with the extracting module and configured to predict a probability of the physiological condition, based on at least one feature.

In another embodiment, the receiving module is configured to receive a plurality of short-term electrocardiogram (ECG) segments of the user. The plurality of short-term ECG segments is recorded at different time points. The stitching module is configured to stitch the plurality of short-term ECG segments for creating a stitched ECG segment. The extracting module is configured to extract at least one feature from the stitched ECG segment, and the predicting module configured to predict the probability of the physiological condition, based on the at least one feature.

In yet another embodiment, the receiving module is configured to receive a plurality of short voice-recordings of the user. The stitching module is configured to stitch the plurality of short voice-recordings for creating a time-series sound data. The extracting module is configured to extract at least one feature from the time-series sound data, and the predicting module is configured to predict an emotion of the user, based on the at least one feature.

In an embodiment of the invention, a method for monitoring a physiological condition of a user is disclosed. The method includes receiving a plurality of short-term Heart Rate Variability (HRV) segments of the user, wherein the plurality of short-term HRV segments is recorded at different time points. The method includes stitching the plurality of short-term HRV segments for creating a stitched HRV segment. The method includes extracting at least one feature from the stitched HRV segment, and predicting a probability of a physiological condition, based on the at least one feature.

To further clarify advantages and features of the disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the disclosure will become better understood when the following detailed description is read with reference to the accompanying drawings in which like characters represent like parts throughout the drawings, wherein:

FIG. 1 illustrates an environment of implementation of a system for monitoring a physiological condition of a user, in accordance with an embodiment of the disclosure;

FIG. 2 illustrates an implementation of the system as illustrated in FIG. 1 in a computing environment, in accordance with an embodiment of the disclosure;

FIG. 3 illustrates an operational flow diagram of the system for monitoring the physiological condition of the user using Heart Rate Variability (HRV) segments, in accordance with an embodiment of the disclosure;

FIG. 4 illustrates an operational flow diagram of the system, for monitoring the physiological condition of the user using electrocardiogram (ECG) segments, in is accordance with an embodiment of the disclosure;

FIG. 5 illustrates an example illustration depicting stitching of ECG segments, in accordance with an embodiment of the disclosure;

FIG. 6 illustrates an operational flow diagram of the system for monitoring the physiological condition of the user using short voice-recordings, in accordance with an embodiment of the disclosure;

FIG. 7 illustrates a flowchart of a method for monitoring a physiological condition of a user using Heart Rate Variability (HRV) segments, according to an embodiment of the present disclosure;

FIG. 8 illustrates a flowchart of the method for monitoring a physiological condition of the user using electrocardiogram (ECG) segments, according to an embodiment of the present disclosure; and

FIG. 9 illustrates a flowchart of the method for monitoring a physiological condition of a user using voice recordings, according to an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the disclosure. Furthermore, in terms of the construction of the device, one or more components of the device may have been represented in the drawings by conventional symbols, and the drawings may show only those specific details that are pertinent to understanding the embodiments of the disclosure so as not to obscure the drawings with details that will be readily apparent to those of ordinary skill in the art having benefit of the description herein.

### DETAILED DESCRIPTION OF FIGURES

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are explanatory of the invention and are not intended to be restrictive thereof

Reference throughout this specification to “an aspect”, “another aspect” or similar language means that a particular feature, structure, or characteristic described in to connection with the embodiment is included in at least one embodiment of the disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are is intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skilled in the art to which this invention belongs. The system, methods, and examples provided herein are illustrative only and not intended to be limiting.

Embodiments of the disclosure will be described below in detail with reference to the accompanying drawings.

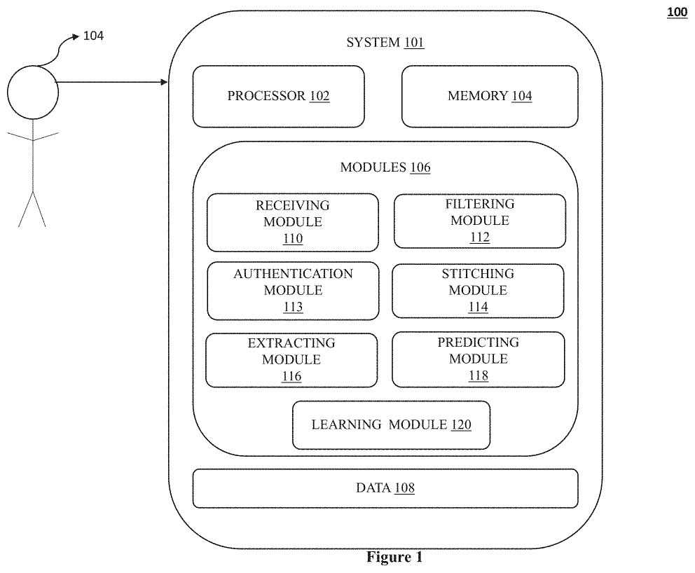

FIG. 1 illustrates an environment 100 for implementing a system 101 for monitoring a physiological condition of a user 104, in accordance with an embodiment of the disclosure. The environment 100 may include the user 104 wirelessly or in a wired connection using electrodes with the system 101. The user 104, which in the present subject matter is a patient, sends signals representative of electrical activity of the heart or voice to recordings to the system 101. The system 101 may be centrally managed or individually addressable to process the signal sent by the user 104 and display text, animation, and video messages for monitoring, prediction, and information, to targeted audiences.

The system 101 may be wirelessly connected to a remote server or a cloud server or the like for accessing various information and getting updated. For the sake of brevity, the remote server or the cloud server has not been shown in the FIG. 1.

The system 101 may include, but is not limited to, a processor 102, a memory 104 and data 108. The system further includes modules 106 which along with the memory 104 may be coupled to the processor 102.

In an embodiment the modules 106 may include a receiving module 110, a filtering module 112, an authentication module 113, a stitching module 114, an extracting module 116, a predicting module 118, and a learning module 120. The receiving module 110, the filtering module 112, the authentication module 113, the stitching module 114, the extracting module 116, the predicting module 118, and the learning module 120 may be in communication with each other. The data 108 serves, amongst other things, as a repository for storing data processed, received, and generated by one or more of the modules 106.

The modules 106, amongst other things, include routines, programs, objects, components, data structures, etc., which perform particular tasks or implement data types. The modules 106 may also be implemented as, signal processor(s), state machine(s), logic circuitries, and/or any other device or component that manipulate signals based on operational instructions.

Further, the modules 106 can be implemented in hardware, instructions executed by a processing unit, or by a combination thereof. The processing unit can comprise a computer, a processor, such as the processor 102, a state machine, a logic array, or any other suitable devices capable of processing instructions. The processing unit can be a general-purpose processor which executes instructions to cause the general-purpose processor to perform the required tasks or, the processing unit can be dedicated to performing the required functions. In another embodiment of the present disclosure, the modules 106 may be machine-readable instructions (software) which, when executed by a processor/processing unit, perform any of the described functionalities.

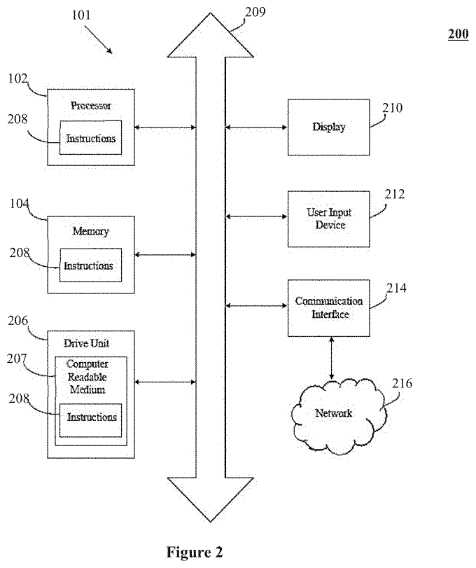

FIG. 2 illustrates an implementation of the system 101 as illustrated in FIG. 1 in a computing environment. The present figure essentially illustrates the hardware configuration of the system 101 in the form of a computer system 200. The computer system 200 can include a set of instructions that can be executed to cause the computer system 200 to perform any one or more of the methods disclosed. The computer system 200 may operate as a standalone device or may be connected, e.g., using a network, to other computer systems or peripheral devices.

In a networked deployment, the computer system 200 may operate in the capacity of a server or as a client user computer in a server-client user network environment, or as a peer computer system in a peer-to-peer (or distributed) network environment. The computer system 200 can also be implemented as or incorporated into various devices, such as a personal computer (PC), a tablet PC, a personal digital assistant (PDA), a mobile device, a palmtop computer, a laptop computer, a desktop computer, a communications device, a wireless telephone, a land-line telephone, a web appliance, a network router, switch or bridge, or any other machine capable of executing a set of instructions (sequential or otherwise) that specify actions to be taken by that machine. Further, while a single computer to system 200 is illustrated, the term “system” shall also be taken to include any collection of systems or sub-systems that individually or jointly execute a set, or multiple sets, of instructions to perform one or more computer functions.

The computer system 200 may include a processor 102 (e, g., a central processing unit (CPU), a graphics processing unit (GPU), or both. The processor 102 may be a component in a variety of systems. For example, the processor 102 may be part of a standard personal computeror a workstation. The processor 102 may be one or more general processors, digital signal processors, application specific integrated circuits, field programmable gate arrays, servers, networks, digital circuits, analog circuits, combinations thereof, or other now known or later developed devices for analyzing and processing data. The processor 102 may implement a software program, such as code generated manually (i.e., programmed).

The computer system 200 may include a memory 104, such as a memory 104 that can communicate via a bus 209. The memory 104 may be a main memory, a static memory, or a dynamic memory. The memory 104 may include but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media, and the like. In one example, the memory 104 includes a cache or random-access memory for the processor 102. In alternative examples, the memory 104 is separate from the processor 102, such as a cache memory of a processor, the system memory, or other memory. The memory 104 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 104 is operable to store instructions executable by the processor 102 The functions, acts or tasks illustrated in the figures or described may be performed by the programmed processor 102 executing the instructions stored in the memory 104. The functions, acts or tasks are independent of the instructions set, storage media, processor or processing strategy and may be performed by software, hardware, integrated circuits, firmware, micro-code and the like, operating alone or in combination. Likewise, processing strategies may include multiprocessing, multitasking, parallel processing, and the like,

As shown, the computer system 200 may or may not further include a display unit 210, such as a liquid crystal display (LCD), an organic light emitting diode (OLED), a. flat panel display, a solid-state display, a cathode ray tube (CRT), a projector, a printer or other now known or later developed display device for outputting determined information. The display 210 may act as an interface for the user to see the functioning of the processor 102, or specifically as an interface with the software stored in the memory 104 or in the drive unit 206.

Additionally, the computer system 200 may include an input device 212 configured to allow a user to interact with any of the components of system 200. The input device 212 may be a number pad, a keyboard, or a cursor control device, such as a mouse, or a joystick, touch screen display, remote control, or any other device operative to interact with the computer system 200.

The computer system 200 may also include a disk or optical drive unit 206. The disk drive unit 206 may include a computer-readable medium 207 in which one or more sets of instructions 208, e.g., software, or modules 106 can be embedded. Further, the instructions 208 may embody one or more of the methods or logic as described. In a to particular example, the instructions 208 may reside completely, or at least partially, within the memory 104 or within the processor 102 during execution by the computer system 200. The memory 104 and the processor 102 also may include computer-readable media as discussed above.

The present invention contemplates a computer-readable median that includes instructions 208 or receives and executes instructions 208 responsive to a propagated signal so that a device connected to a network 216 can communicate voice, video, audio, images or any other data over the network 216. Further, the instructions 208 may be transmitted or received over the network 216 via a communication port or interface 214 or using a bus 209. The communication port or interface 214 may be a part of the processor 102 or may be a separate component. The communication port 214 may be created in software or may be a physical connection in hardware. The communication port 214 may be configured to connect with a network 216, external media, the display 210, or any other components in computer system 200 or combinations thereof. The connection with the network 216 may be a physical connection, such as a wired Ethernet connection or may be established. wirelessly as discussed later. Likewise, the additional connections with other components of the computer system 200 may be physical connections or may be established wirelessly. The network 216 may alternatively be directly connected to the bus 209.

The network 216 may include wired networks, wireless networks, Ethernet AVB networks, or combinations thereof. The wireless network may he a cellular telephone network, an 802.11, 802.16, 802.20, 802.1Q or WiMax network. Further, the network 216 may be a public network, such as the Internet, a private network, such as an intranet, or combinations thereof, and may utilize a variety of networking protocols now available or later developed including, but not limited to TCP/IP based networking protocols.

In an example, dedicated hardware implementations, such as application specific integrated circuits, programmable logic arrays and other hardware devices, can be constructed to implement various parts of the system 101. A detailed implementation of the system 100 will be explained in the forthcoming paragraphs.

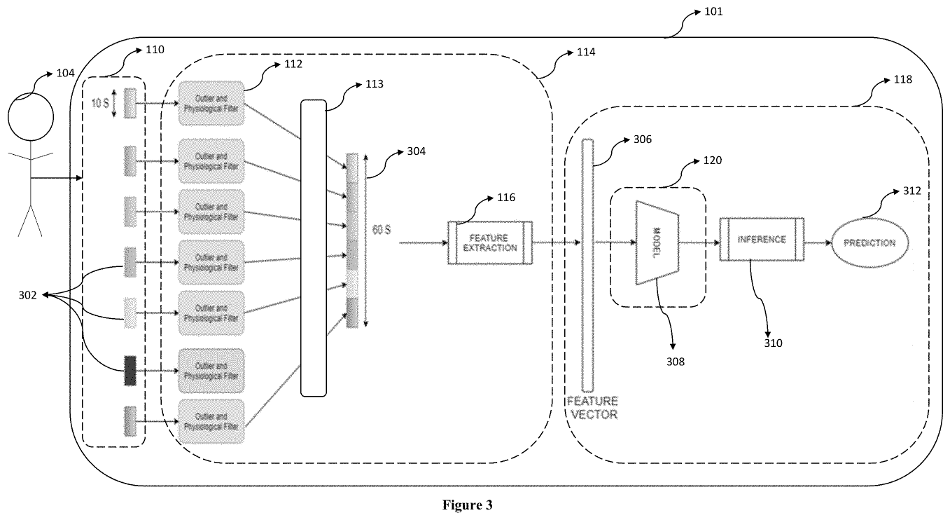

FIG. 3 illustrates an operational flow diagram of the system 101, for is monitoring the physiological condition of the user 104 using Heart Rate Variability (HRV) segments, in accordance with an embodiment of the disclosure. Referring to FIG. 1, FIG. 2, and FIG. 3, the receiving module 110 may be configured to receive a plurality of short-term Heart Rate Variability (HRV) segments 302 from the user 104. The short-term HRV segments 302 may be recorded at different time points throughout a day. As an example, the short-term HRV segments 302 are multiple 10 seconds segments recorded at multiple time intervals. The receiving module 110 may be in communication with the filtering module 112.

The filtering module 112 may be a noise-based filtering module. As an example, the short-term HRV segments 302 are filtered using the noise-based filtering module 112 based on a predefined quality index to maintain quality of the combined segment. The short-term HRV segments 302 are scrutinized with the noise-based filtering module 112 using relevant quality indices based on their properties, e.g., physiological and noise-based filtering. Therefore, the filtering module 112 may select the short-term HRV segments 302 having a quality score higher than the predefined quality index, for further processing. The filtering module 112 may be in communication with the authentication module 113.

The authentication module 113 may be configured to receive the short-term to HRV segments 302. Further, the authentication module 113 may identify the plurality of short-term HRV segments 302 associated with the same user 104. The authentication module 113 authenticates that the plurality of short-term HRV segments 302 related to/are of the same user 104 and does not allow any other user's short-term HRV segments to be mixed. Thus, the authentication module 113 ensures that the data used for further processing in the next steps belongs to the same user 104. The authentication module 113 may be in communication with the stitching module 114.

The stitching module 114 may be configured to receive the short-term HRV segments 302. Further, the stitching module 114 may stitch the plurality of short-term HRV segments 302 for creating a stitched HRV segment 304. As an example, the stitched HRV segment 304 is of N*P length, wherein ‘N’ is indicative of a number of short-term HRV segments 302, and ‘P’ is indicative of a length of each short-term HRV segment 302. In the example, the stitched HRV segment 304 is combination of 10 s short-term HRV segments 302 stitched together, resulting in formation of a 60 s stitched HRV segment 304. Further, the stitching module 114 may be in communication with the extracting module 116.

The extracting module 116 may be configured to extract at least one feature from the stitched HRV segment 304. The feature extracted from the stitched HRV segment 304 may include a greater degree of variance arising from the shift in physiological condition of the user 104 during different time points of recording. This increased variance thus brings in more information of the physiological condition of the user 104 as compared to short-term HRV segments 302. The stitched HRV segment 304 contains more information/features as compared to individual short-term HRV segments 302 as it is exposed to longer trends in data. The extracting module 116 may be in communication with the predicting module 118.

In an embodiment, the features extracted are converted into a feature vector 306 and passed to the predicting module 118. The predicting module 118 may be configured to predict 312 a probability of the physiological condition, based on the extracted feature. In an example embodiment, processing of the short-term HRV segments 302 results in predicting the physiological condition of hypertension in the user 104. Thus, by processing the electric signal from the user 104, the system is capable to monitor the physiological condition.

In an embodiment, the feature vector 306 is passed through the learning module 120. The learning module 120 may be adapted to train a neural network model 308. In an embodiment, the learning module 120 may train the neural network model 308 based on the extracted feature. The learning module 120 may be in communication with the predicting module 118. The predicting module 118 may configured to predict the probability of the physiological condition based on the learning of the model.

As the stitched HRV segment 304 increases the amount of information available to the neural network model 308, it improves these predictions of the physiological condition with each prediction. Additionally, the variance seen by the neural network model 308 is increased by the stitched HRV segment 304 and helps the neural network model 308 achieve better accuracy and comparable performance to similar length continuous segments.

The parsing of feature vector 306 through the learning module 120 trains the neural network model 308 in prediction of the physiological condition with better accuracy. The inference module 310 is used to infer the physiological descriptors of the user 104 and form a prediction 312 of the condition of the user 104.

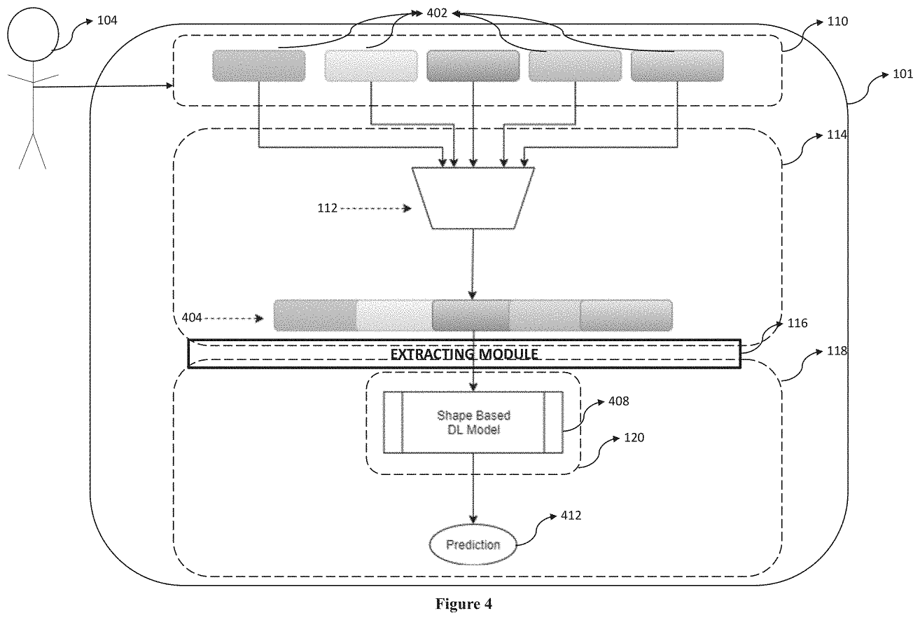

FIG. 4 illustrates an operational flow diagram of the system 101, for monitoring the physiological condition of the user 104 using electrocardiogram (ECG) segments. Referring to FIG. 1, FIG. 2, and FIG. 4, the receiving module 110 may be configured to receive a plurality short-term electrocardiogram (ECG) segments 402 from the user 104. The short-term ECG segments 402 may be recorded at different time points, and the short-term ECG segments 402 may be filtered by the filtering module 112 for further processing. As an example, the short-term ECG segments 402 are filtered using the noise-based filtering module 112 based on a predefined quality index to maintain qualify of the combined segment.

Subsequently, the stitching module 114 may receive the filtered short-term ECG segments 402. The stitching module 114 may stitch the selected short-term ECG segments 402 for creating a stitched ECG segment 404. As an example, the stitched ECG segment 404 is of N*P length, wherein ‘N’ is indicative of a number of short-term ECG segment 402, and ‘P’ is indicative of a length of each short-term ECG segment 402.

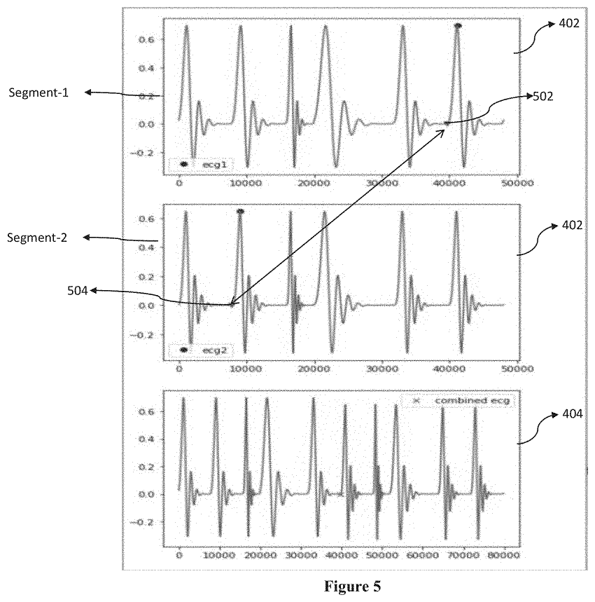

FIG. 5 illustrates an example illustration depicting stitching of the filtered ECG segments. Continuing with further description of FIG. 4, the FIG. 5 presents two short-term ECG segments 402. In the stitching module 114, two short-term ECG segments 402 are stitched with precision to remove as much irregularity at point of stitching. The last Zero-crossing point 502 before the last Peak of segment-1 is matched with the last Zero-crossing point 504 before the first Peak of segment-2.

Further, the extracting module 116 may extract at least one feature from the stitched ECG segment 404. The features extracted are passed to the predicting module 118 to predict 412 a probability of the physiological condition, based on at least one feature. In an example embodiment, processing of the short-term ECG segments 402 results in predicting the physiological condition of hypertension in the user 104. Thus, by processing the ECG signals from the user 104, the system 101 is capable to monitor the physiological condition.

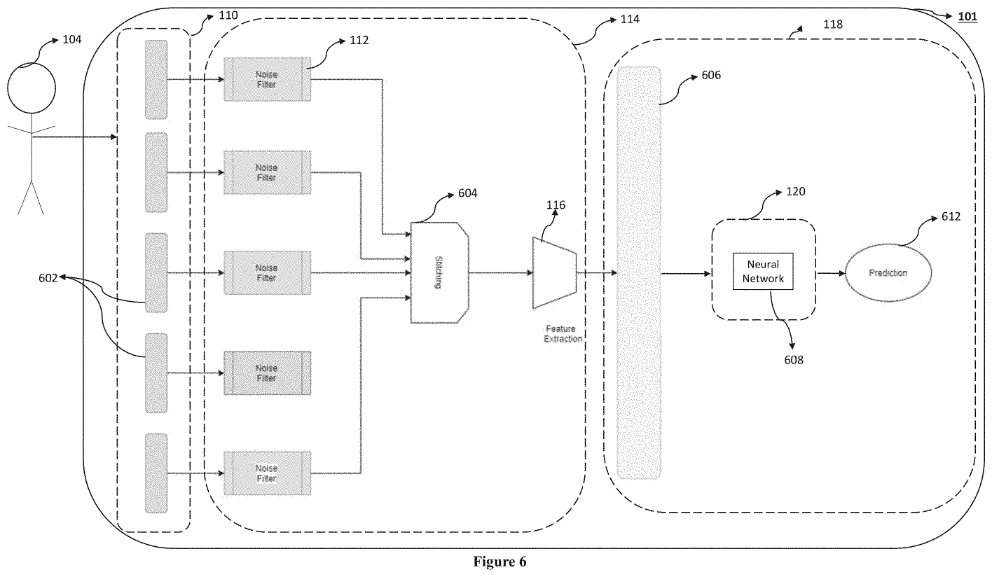

FIG. 6 illustrates an operational flow diagram of the system 101, for monitoring the physiological condition of the user 104 using short voice-recordings. Referring to FIG. 1, FIG. 2, and FIG. 6, the receiving module 110 may be configured to receive a plurality short voice-recordings 602 from the user 104. The short voice-recordings 602 may be recorded at different time points, and the short voice-recordings 602 may be filtered by the filtering module 112 for further processing. As an example, the short voice-recordings 602 are filtered using the noise-based filtering module 112 based on a predefined quality index to maintain quality of the combined segment.

Subsequently, the stitching module 114 may receive the short voice-recordings 602. The stitching module 114 may stitch the selected short voice-recordings 602 for creating a stitched time-series sound data 604. As an example, time series sound data 604 of N*P length, wherein is indicative of a number of voice recordings 602 and ‘P’ is indicative of a length of each voice recording 602.

Further, in the present disclosure the extracting module 116 in communication with the stitching module 114, extracts at least one feature from the time series sound data 604. The features extracted are passed to the prediction module 118 in communication with the extracting module 116 and configured to predict 612 a mood or emotion of the user 104, based on at least one feature. In an example embodiment, processing of the short voice-recordings 602 results in predicting the emotional state of the user 104. Thus, by processing to the voice recordings from the user 104, the system is capable to monitor the mood of the user 104.

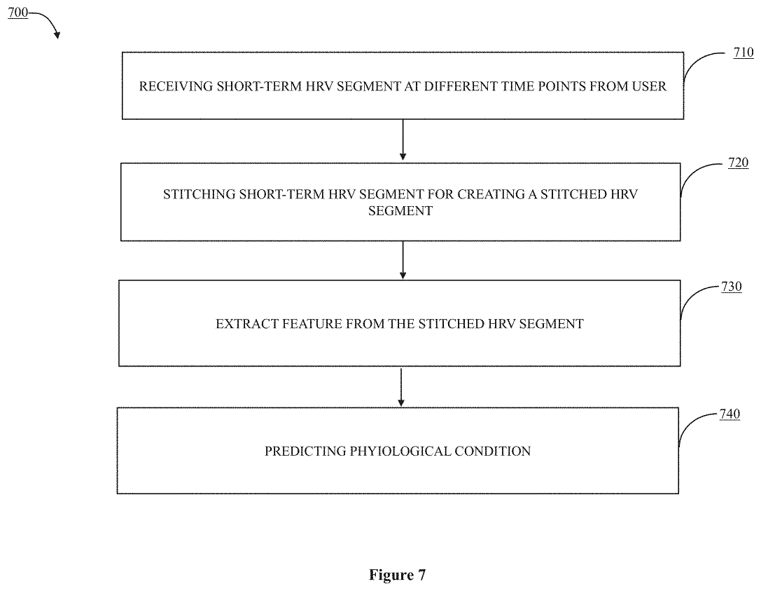

FIG. 7 illustrates a flowchart of a method 700 for monitoring a physiological condition of the user using Heart Rate Variability (HRV) segments, according to an embodiment of the present disclosure. The method 700 may be implemented by the system 101 using components thereof, as described above. In an embodiment, the method 700 may be executed by the receiving module 110, the stitching module 114, the extracting module 116 and the predicting module 118 and components therein. Further, for the sake of brevity, details of the present disclosure that are explained in detail in the description of FIG. 1-FIG. 6 are not disclosed herein.

At block 710, the method 700 includes receiving short-term FV segments at different time points from the user.

At block 720, the short-term HRV segments are stitched to form a stitched HRV segment as a longer continuous signal.

Thereafter, at block 730, the method 700 includes extracting feature from the stitched HRV segment.

Further, at block 740 the method 700 analyses the stitched HRV segment for predicting the physiological condition of the user.

In an embodiment, the features extracted are used by the neural network model for training and predicting the physiological condition of the user with accuracy.

In an embodiment of the invention, the method 700 further includes selecting short-term segments based on predefined quality index. In particular, the multiple short-term segments recorded from the user at different time points are subjected to filtering based on properties, e.g., physiological, shape-based, and noise-based filtering.

In an embodiment of the invention, the method 700 further includes authenticating the plurality of short-term HRV segments associated with the same user. In particular, the plurality of short-term HRV segments recorded from the user are subjected to authentication to ensure that the short-term HRV segments are related to the same user. is Thus, the method 700 ensures that the data used for further processing in the next steps belongs to the same user.

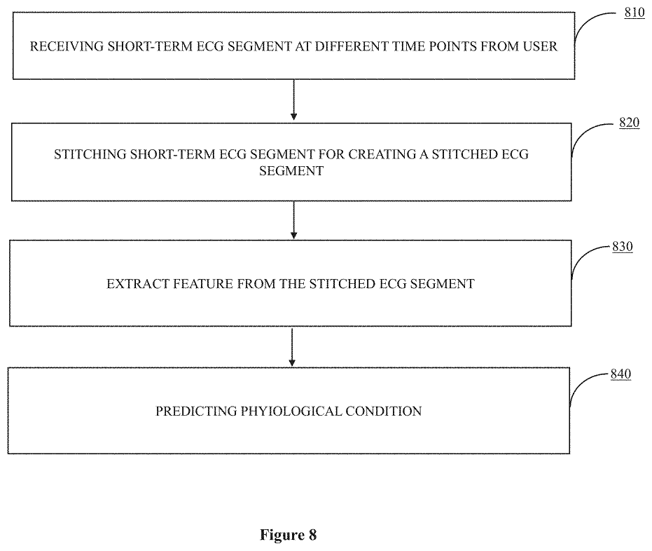

FIG. 8 illustrates a flowchart, of the method 700 for monitoring a physiological condition of the user using electrocardiogram (ECG) segments, according to an alternative embodiment of the present disclosure. At block 810, an alternative embodiment of the method 700 includes receiving short-term ECG segments at different time points from the user.

At block 820, the short-term ECG segments are stitched to form a stitched ECG segment as a longer continuous signal.

Thereafter, at block 830, the method 700 includes extracting feature from the stitched ECG sement

Further, at block 840 the method 700 analyses the stitched ECG segment for predicting the physiological condition of the user.

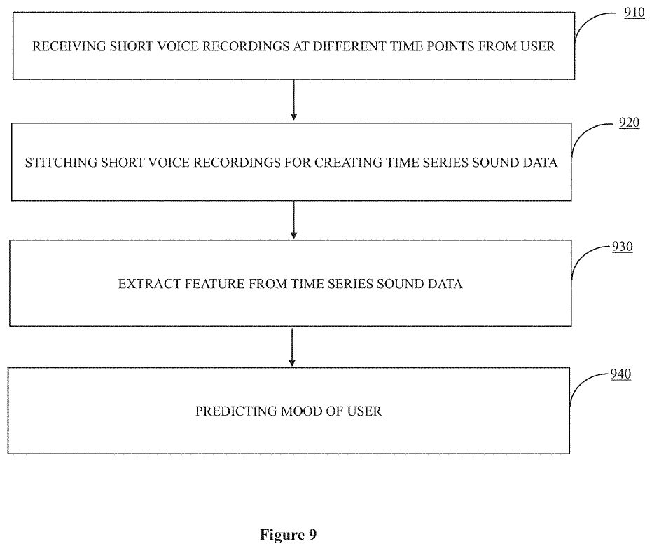

FIG. 9 illustrates a flowchart, of the method 700 for monitoring a physiological condition of a user using voice recordings, according to an alternative embodiment of the present disclosure. At block 910, an alternative embodiment of the method 700 includes u receiving short voice recordings from the user,

At block 920, the short voice recordings are stitched to form a stitched time series sound data as a longer continuous signal.

Thereafter, at block 930, the method 700 includes extracting feature from the time series sound data.

Further, at block 940 the method 700 analyses the time series sound data for predicting the physiological condition of the user.

In an advantage of the present subject matter, the stitching of the short-term HRV segments 302, stitching of the short-term ECG segments 402, and stitching of the short voice recordings 602 results in increasing the amount of information available to the neural network model (308, 408, 608) to improve the predictions of the physiological condition and provide the system 101 capability to monitor the physiological condition of the user 104. The selection of the short-term signals at different time points and then selecting the short-term signals through the filtering module 112, provides a greater degree of variance to the neural network model (308, 408, 608) and helps model achieve comparable performance to similar length continuous segments.

Terms used in this disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, to such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits is any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted. to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description of embodiments, claims, or drawings, should be understood to contemplate the possibilities of including one of the terms, either of the terms or both terms. For example, the phrase “A or B” should be understood to include the possibilities of “A” to or “B” or “A and B.”

All examples and conditional language recited in this disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that various changes, substitutions, and alterations could be made thereto without departing from the spirit and scope of the present disclosure.

Moreover, the actions of any flow diagram need not be implemented in the order shown; nor do all of the acts necessarily need to he performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the present subject matter, any limitations arising on account thereto, are not intended. As would be apparent to a to person in the art, various working modifications may be made to the method in order to implement the inventive concept as taught herein. The drawings and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment.

1. A system (101) for monitoring a physiological condition of a user (104), the system comprising:
a receiving module (110) configured to receive a plurality of short-term Heart Rate Variability (HRV) segments (302) of the user (104), wherein the plurality of short-term HRV segments (302) is recorded at different time points;
a stitching module (114) in communication with the receiving module (110) and configured to stitch the plurality of short-term HRV segments (302) for creating a stitched HRV segment (304);
an extracting module (116) in communication with the stitching module (114) and configured to extract at least one feature from the stitched HRV segment (304); and
a predicting module (118) in communication with the extracting module (116) and configured to predict (312) a probability of the physiological condition, based on at least one feature.

2. The system (101) as claimed in claim 1, comprising a noise-based filtering module (112) in communication with the receiving module (110) and configured to select the plurality of short-term HRV segments (302), based on a predefined quality index, to generate the stitched HRV segment (304).

3. The system (101) as claimed in claim 2, comprising an authentication module (113) in communication with the filtering module (112) and configured to authenticate the plurality of short-term HRV segments (302) associated with the user (104).

4. The system (101) as claimed in claim 1, wherein the stitching module (114) is configured to create the stitched HRV segment (304) of N*P length, wherein ‘N’ is indicative of a number of short-term HRV segments (302) and ‘P’ is indicative of a length of each short-term HRV segment (302).

5. The system (101) as claimed in claim 1, comprising:
the receiving module (110) configured to receive a plurality of short-term electrocardiogram (ECG) segments (402) of the user (104), wherein the plurality of short-term ECG segments (402) is recorded at different time points;
the stitching module (114) configured to stitch the plurality of short-term ECG to segments (402) for creating a stitched ECG segment (404);
the extracting module (116) configured to extract at least one feature from the stitched ECG segment (404); and
the predicting module (118) configured to predict (412) the probability of the physiological condition, based on the at least one feature.

6. The system (101) as claimed in claim 5, further comprising the noise-based filtering module (112) configured to select the plurality of short-term ECG segments (402), based on a predefined quality index, to generate the stitched ECG segment (404).

7. The system (101) as claimed in claim 5, wherein the stitching module (114) is configured to create the stitched ECG segment (404) of MP length, wherein ‘N’ is indicative of a. number of short-term ECG segments (402) and ‘P’ is indicative of a length of each short-term ECG segment (402).

8. The system (101) as claimed in claim 5, comprising:
the learning module (120) configured to train a neural network model (408) based on the at least one feature; and
the predicting module (118) configured to predict the probability of the physiological condition based on the learning of the model.

9. The system (101) as claimed in claim 1, comprising:
the receiving module (110) configured to receive a plurality of short voice-recordings (602) of the user (104);
the stitching module (114) configured to stitch the plurality of short voice-recordings (602) for creating a time-series sound data (604);
the extracting module (116) configured to extract at least one feature from the time-series sound data (604); and
the predicting module (118) configured to predict a mood of the user (104), based on the at least one feature,

10. The system (101) as claimed in claim 9. comprising the noise-based filtering module (112) to select the plurality of short voice recordings (602), based on a predefined quality index, to generate the time series sound data (604).

11. The system (101) as claimed in claim 9, wherein the stitching module (114) is configured to create the time series sound data (604) of MP length, wherein ‘N’ is indicative of a. number of voice recordings (602) and ‘P’ is indicative of a length of each voice recording (602).

12. A method (700) for monitoring a physiological condition of a user, the method comprising:
receiving (710) a plurality of short-term Heart Rate Variability (HRV) segments of the user, wherein the plurality of short-term HRV segments is recorded at different time points;
stitching (720) the plurality of short-term HRV segments for creating a stitched HRV segment;
extracting (730) at least one feature from the stitched HRV segment; and
predicting (740) a probability of a physiological condition, based on the at least one feature.

13. The method (700) as claimed in claim 12, comprising:
selecting the plurality of short-term HRV segments, based on a predefined quality index, for generating the stitched HRV segment.

14. The method (700) as claimed in claim 13, comprising: authenticating the plurality of short-term HRV segments, based on association with the user.

15. The method (700) as claimed in claim 12, comprising:
receiving (810) a plurality of short-term electrocardiogram (ECG) sements of the user, wherein the plurality of short-term ECG segments is recorded at different time points;
stitching (820) the plurality of short-term ECG segments for creating a stitched ECG segment;
extracting (830) at least one feature from the stitched ECG segment;
predicting (840) the probability of the physiological condition, based on the at least one feature.

16. The method (700) as claimed in claim 15, comprising:
selecting the plurality of short-term ECG segments, based on a predefined quality index, for generating the stitched ECG segment.

17. The method (700) as claimed in claim 12, comprising:
receiving (910) a plurality of short voice-recordings of the user;
to stitching (920) the plurality of short voice-recordings for creating a time-series sound data;
extracting (930) at least one feature from the time-series sound data; and
predicting (940) a mood of the user, based on the at least one feature.

18. The method (700) as claimed in claim 17, comprising:
selecting the plurality of short voice recordings, based on a predefined quality index, to generate the time series sound data.
